(19) United States
(12) Patent Application Publication
(10) Pub. No.: US 2025/0006378 A1
(43) Pub. Date: Jan. 2, 2025

(54) SYSTEM AND METHOD FOR EVALUATING A CARDIAC REGION OF A SUBJECT

(71) Applicant: FEops NV, Gent (BE)

(72) Inventors: Peter Eddy J. MORTIER, Ingooigem (BE); Maxime NAUWYNCK, Brugge (BE); Kilian MICHIELS, Oostduinkerke (BE); Eva HEFFINCK, Kortrijk (BE); Giorgia ROCATELLO, Zwijnaarde (BE)

(73) Assignee: FEops NV, Gent (BE)

(21) Appl. No.: 18/343,662

(22) Filed: Jun. 28, 2023

(51) Int. Cl. G16H 50/30; A61B 5/02; A61B 5/107; A61B 34/10; G16H 40/40; G16H 40/63; G16H 50/20; G16H 50/50

(52) U.S. Cl. CPC G16H 50/30; A61B 5/02007; A61B 5/1075; A61B 34/10; G16H 40/40; G16H 40/63; G16H 50/20; G16H 50/50; A61B 2034/102; A61B 2034/105; G16H 10/60; G16H 30/20

(57) ABSTRACT

A system and method for evaluating a cardiac region of a subject from medical data. The method comprises obtaining, by the computer system, for a sequence of positions along a centerline of the coronary artery a sequence of associated luminal dimensions. The method comprises determining, by the computer system, data relating to a Fractional Flow Reserve, FFR, of the coronary artery by comparing the sequence of luminal dimensions of the coronary artery of the subject with one or more reference sequences of luminal dimensions. The method further comprises displaying the data relating to the FFR on a display.

### FIELD OF THE INVENTION

The present invention relates to the field of evaluating a cardiac region of a subject. In particular, the invention relates to the field of evaluating a coronary artery of a subject from medical data. More in particular, the invention relates to a system and method for determining data relating to a Fractional Flow Reserve of a coronary artery of a subject from medical data.

### BACKGROUND

It is known to use diagnostic methods in order to detect heart disease. Physicians may diagnose patients with chest pain with heart disease after examining the cardiac region of the patient. Detecting obstructive coronary artery disease can involve measuring coronary pressure by inserting a pressure sensor in a coronary artery by an interventional cardiologist. From the difference in coronary pressure upstream and downstream of a coronary artery obstruction, the cardiologist can determine a Fractional Flow Reserve, FFR, of the coronary artery of the patient. The FFR is commonly defined as the ratio of the coronary pressures measured directly distal and proximal of a stenosis. The FFR is a dimensionless number in the numerical range from 0 (complete occlusion) through 1 (no occlusion). An FFR-value below 0.75-0.80 is generally considered to reflect a clinically significant stenosis. The FFR value of the coronary artery can aid a cardiologist in determining a treatment plan suitable for the patient. It is also known that different FFR values require different treatment methods, such as treatment by drugs, therapy and/or surgery. Nevertheless, cardiac procedures of inserting a pressure sensor in a coronary artery by a cardiologist are uncomfortable, cumbersome and can provide health risks to the patient. For cardiologists, detecting coronary artery obstructions by invasive coronary pressure measurement are labour intensive.

### SUMMARY

It is an object to provide a method for evaluating a cardiac region of a subject, a method for assisting in selecting a preferred stent for implantation, a method of classifying a sequence of luminal dimensions of a coronary artery in order to determine a FFR classification of the coronary artery of a subject and/or an electronic image processing system. More in general, it is an object to provide an improved electronic image processing system and/or method for evaluating a cardiac region of a subject.

Thereto, according to a first aspect is provided a computer-implemented method for evaluating a cardiac region of a subject from medical data. The method comprises in step a) obtaining, by the computer system, for a sequence of positions along a centerline of the coronary artery a sequence of associated luminal dimensions. The sequences can each be formed by an array of numerical values. The sequences can be fed to the computer system in step a). The coronary artery can be computationally divided into a plurality of segments along the centerline. The coronary artery can be divided into segments at regular intervals, wherein the intervals are optionally evenly distributed. The intervals of the segments together indicate the sequence of positions along the centerline of the coronary artery. At least a part of the positions along the centerline of the coronary artery can therefore be equally spaced along the respective part of the centerline. The luminal dimensions can be determined, e.g. computed, for each segment. The luminal dimensions of the segments together indicate the sequence of luminal dimensions associated with the sequence of positions along the centerline of the coronary artery. The method comprises in step b1) determining, by the computer system, data relating to a Fractional Flow Reserve, FFR, of the coronary artery by comparing the sequence of luminal dimensions of the coronary artery of the subject with one or more reference sequences of luminal dimensions. The FFR is commonly defined as the ratio of the coronary pressures directly distal and proximal of a stenosis. Herein, the FFR can also be defined for a section of a coronary artery, as the ratio of the coronary pressures directly distal and proximal of the section. Herein, the FFR can also be defined for an entire coronary artery, as the ratio of the coronary pressures directly distal and proximal of the coronary artery. Herein, the FFR can also be defined for a segment of a coronary artery, as the ratio of the coronary pressures directly distal and proximal of the segment. The reference sequences of luminal dimensions can be obtained using a computer system and/or by coronary pressure measurement. The reference sequences of luminal dimensions can be obtained from medical data from other coronary arteries of the same subject or from medical data of other subjects. The reference sequences of luminal dimensions can be obtained by constructing an anatomic model of coronary arteries. The anatomic model comprises the at least one coronary artery, and can be constructed by the computer system. Determining the data relating to the FFR can comprise determining a single FFR value, or determining a sequence of progressive or local FFR's, for the sequence of luminal dimensions. The method comprises in step c) displaying the data relating to the FFR on a display.

According to a second aspect is provided a computer-implemented method for evaluating a cardiac region of a subject from medical data. The method comprises in step a) obtaining, by the computer system, for a sequence of positions along a centerline of the coronary artery a sequence of associated luminal dimensions. The sequences can each be formed by an array of numerical values. The sequences can be fed to the computer system in step a). The coronary artery can be computationally divided into a plurality of segments along the centerline. The coronary artery can be divided into segments at regular intervals, wherein the intervals are optionally evenly distributed. The intervals of the segments together indicate the sequence of positions along the centerline of the coronary artery. At least a part of the positions along the centerline of the coronary artery can therefore be equally spaced along the respective part of the centerline. The luminal dimensions can be determined, e.g. computed, for each segment. The luminal dimensions of the segments together indicate the sequence of luminal dimensions associated with the sequence of positions along the centerline of the coronary artery. The method comprises in step b2) determining, by the computer system, data relating to a Fractional Flow Reserve, FFR, of the coronary artery from the sequence of luminal dimensions using a trained machine learning data processing model. The trained machine learning data processing model is trained to determine data relating to an FFR based on a sequence of luminal dimensions. The sequence of luminal dimensions used for training the machine learning data processing model can be obtained using a computer system and/or by coronary pressure measurement. The luminal dimensions used for training the machine learning model can be obtained from medical data from other coronary arteries of the same subject or from medical data of other subjects. The reference sequences of luminal dimensions can be obtained by constructing an anatomic model of coronary arteries. The anatomic model comprises the at least one coronary artery, and can be constructed by the computer system. Determining the data relating to the FFR can comprise determining a single FFR value, or determining a sequence of progressive or local FFR's, for the sequence of luminal dimensions. The method comprises in step c) displaying the data relating to the FFR on a display.

For both aspects the following applies.

Optionally, a single FFR is determined for the sequence of luminal dimensions. The single FFR can be determined for the entire sequence of luminal dimensions of the coronary artery. Hence the single FFR reflects the entire coronary artery under study. A single FFR can be calculated for the total of all obstructions in the coronary artery associated with the positions included in the sequence of positions along the length of the coronary artery.

Optionally, a sequence of local FFR's is determined for the sequence of luminal dimensions. A local FFR value can e.g. be computed for each obstruction in the coronary artery, wherein more than one obstruction can occur in a single coronary artery. A local FFR value can e.g. be computed for each position of the sequence of positions along the length of the coronary artery.

Optionally, a sequence of progressive FFR's is determined for the sequence of luminal dimensions. A progressive FFR value can e.g. be computed for each position of the sequence of positions along the length of the coronary artery, wherein each progressive FFR value reflects the ratio of the coronary pressure directly distal of the respective position and proximal of the (start of the) coronary artery.

Optionally, the luminal dimensions comprise one or more of diameter, average diameter, minimum diameter, maximum diameter, volume, cross sectional area, and curvature radius of the coronary artery. One or more of the luminal dimension parameters can comprise similar values for luminal dimensions associated with different positions along the length of the coronary artery. The maximum diameter can comprise a diameter of a circle circumscribing the local cross section of the coronary artery. The minimum diameter can comprise a diameter of a largest circle fitting in the local cross section of the coronary artery.

Optionally, the luminal dimensions further can comprise an indication of the presence of plaque and at least one of size, location and severity of the plaque. The indication can be explicitly indicated by i.e. a separate parameter relating to the plaque presence. An implicit indication of the presence of plaque is possible by e.g. at least partially excluding the plaque area from a determined luminal dimension. The method can further comprise identifying a region of interest in the medical image data, wherein the region of interest comprises one or more coronary arteries optionally including the presence of plaque.

Optionally, the steps b1 and/or b2 of determining data relating to the FFR include patient data, such as age, gender, weight and/or length, as input. By including patient data as input, the data relating to the FFR can be determined more accurately due to more tailored comparison (step b1) and/or prediction (step b2) of the luminal dimensions. The patient data can aid in selecting the relevant reference sequences of luminal dimensions of the coronary arteries having a similar condition as the luminal dimensions currently under investigation. By taking into account the patient data in comparing (step b1) or predicting (step b2), the accuracy of the determined data relating to the FFR can increase.

Optionally, the method further comprising, e.g. automatically, determining, by the computer system, the sequence of luminal dimensions from medical image data. When directly determining the luminal dimensions from medical image data, no anatomical model is required to be constructed. The sequence of luminal dimensions can be determined from the medical image data using image recognition methods. Using a trained machine learning model, the luminal dimensions can be automatically determined by the computer system.

Optionally, the medical image data is obtained from one or more Computed Tomography, CT, images, and/or Magnetic Resonance Imaging, MRI, images and/or fluoroscopic images. The medical images can be pre-processed prior to, during, or after obtaining medical data therefrom.

Optionally, the method further comprises determining, prior to step b1 or b2, a modified sequence of luminal dimensions, by substituting, for at least some of the positions along the centerline, a determined luminal dimension of the coronary artery by a luminal dimension of a deployed stent. The data relating to the FFR subsequently determined in step b1 or b2 then can predict a value of the data relating to the FFR in case of said stent being computationally deployed in the coronary artery. A deployed stent can be a stent that has been computationally inserted into the coronary artery and is expanded in the coronary artery at the indicated location. The deployed stent can be computed as positioned at its end location.

According to a third aspect is provided a computer-implemented method for assisting in selecting a preferred stent for implantation. The method comprises in step d) for a plurality of stent sizes, types, and/or locations in a coronary artery, performing the method as described comprising determining a modified sequence of luminal dimensions, by substituting some luminal dimensions by a luminal dimension of a deployed stent. The luminal dimensions that are substituted by a luminal dimension of a deployed stent are associated with similar positions along the centerline of the coronary artery as the luminal dimensions of the deployed stent. The method further comprises in step e) for each of the plurality of stent sizes, types, and/or locations displaying the data relating to the FFR on a display. The data relating to the FFR of the different stent sizes, types, and/or locations can be displayed on one part of the display or on separate parts of the display, such as separate windows or separate columns of a table.

Optionally, the method further comprises the computer selecting a preferred stent size, type, and/or location on the basis of the data relating to the FFR for each of the plurality of stent sizes, types, and/or locations determined in step d). A preferred stent size, type, and/or location may e.g. be chosen that results in the highest FFR value for the entire coronary artery or part thereof, or the highest local or progressive FFR value, or the largest positive change in FFR value e.g. at a stenosis. It will be appreciated that the computer-implemented method relates to virtual stent implantation. It will also be appreciated that based on performing the computer-implemented method, a real-life stent may be implanted in the real-life patient, e.g. the selected stent size and type at the selected location.

Optionally, the method comprises for a single stent size and type, performing the method as described, comprising determining a modified sequence of luminal dimensions, and comprising steps d) and e), for virtual placement of the stent in the coronary artery at a plurality of different locations along the entire sequence of positions along the centerline. Virtual stent placement refers to the end location of the stent, i.e. the position of the stent after deployment in the coronary artery. The placement of the stent herein does not refer to the process of virtually moving the stent into the subject to the desired position. Stent placement is therefore concerning a final stent destination. The plurality of different locations can be associated with different coronary artery obstructions. The method further comprises displaying an indication representative of a value of the data relating to the FFR associated with placement of the stent at said locations along the sequence of positions on a display. For a single stent type and location, the method for stent placement as described can alternatively be performed for multiple different sizes of stents in step d). Alternatively, for a single stent size and location, the method for stent placement as described can be performed for multiple different types of stents in step d).

Optionally, the method further comprises displaying the stent on a three-dimensional coronary model on the display. The three-dimensional model can be determined based on the medical image data and/or a computed anatomic model.

Optionally, the displaying further comprises displaying image features and/or parameter values on the display. The image features can be shown on the three-dimensional model. The parameter values can be displayed in a table.

Optionally, the method further comprises manipulating the view and/or displayed parameter values of the coronary model. The view and/or parameter values can be manipulated by a user through e.g. a user interface.

Optionally, the method further comprises automatically changing the view and/or displayed parameter values of the coronary model according to the stent number, size and/or location. The view and/or displayed parameter values of the coronary model can be automatically changed for improved ease of access to the displayed information by a user, such as a physician or cardiologist.

Optionally, the method comprises determining an optimal viewing angle of the three-dimensional coronary model for optimally visualizing the stent portion of the three-dimensional coronary model associated with the placement of the stent. The optimal viewing angle can correspond to an optimal c-arm position for e.g. fluoroscopic imaging of the cardiac region of the subject, such as during actual insertion of an actual stent in the patient's body.

Optionally, the method further comprising recording a position of a C-arm of a X-ray imaging unit that corresponds to the optimal viewing angle.

Optionally, the position of the C-arm corresponding to the optimal viewing angle is established automatically.

According to a fourth aspect is provided, in an electronic image processing system, a method of classifying a sequence of luminal dimensions of a coronary artery in order to determine a Fractional Flow Reserve, FFR, classification of the coronary artery of a subject by using a trained machine learning data processing model. The trained machine learning data processing model is trained to classify the sequence of luminal dimensions as representing an FFR value according to at least a first class of FFR values or a second class of FFR values. The first class can e.g. correspond to FFR values greater than 0.8, and the second class can e.g. correspond to FFR values smaller than or equal to 0.8. It will be appreciated that the trained machine learning model can be trained to classify the FFR values according to more than two classes of FFR values. The method comprises receiving, by the trained machine learning data processing model, the sequence of luminal dimensions. The method further comprises determining, using the trained machine learning data processing model, to which of the at least the first and second class of FFR values the sequence of luminal dimensions corresponds. The method comprises providing an indication of the class of FFR values to which the sequence of luminal dimensions corresponds. The indication can either comprise an indication of the first class of FFR values or an indication of the second class of FFR values for said coronary artery. It will be appreciated that an indication of more possible classes of FFR values can be provided corresponding to the number of classes provided in training of the machine learning model. Optionally, a level of certainty is indicated of the classification of the sequence of luminal dimensions as representing an FFR value according to the selected class of FFR values.

Optionally, the sequence of luminal dimensions comprises at least one of diameter, average diameter, minimum diameter, maximum diameter, volume, cross sectional area, and curvature radius of the coronary artery. Further, the sequence of luminal dimensions optionally comprises an indication of the presence of plaque and at least one of size, location and severity of plaque.

According to a fifth aspect is provided a method of training a machine learning data processing model for performing a method of classifying a sequence of luminal dimensions of a coronary artery in order to determine a FFR classification of the coronary artery of a subject as described. The method of training comprises in step a) receiving, by the machine learning data processing model, a training data set comprising training data, the training data including a plurality of known sequences of luminal dimensions along coronary artery locations. The training data set comprises training data, and the training data includes a plurality of known sequences of luminal dimensions along coronary artery locations. The known sequences of luminal dimensions can be from other coronary arteries of the same subject or from other subjects. The training method comprises in step b) receiving, by the machine learning data processing model, a ground truth data set comprising ground truth data indicative of at least a first or second FFR class associated with the training data. The training method comprises training the machine learning data processing model in step c). The machine learning data processing model is trained in step c) based on the training data received in step a), and the ground truth data received in step b), for enabling the machine learning data processing model, after completion of the training period, to perform the step of classifying the sequence of luminal dimensions as representing an FFR value according to one of at least a first class of FFR values or a second class of FFR values. It will be appreciated that the machine learning model can be trained to classify the FFR values according to more than two classes of FFR values.

Optionally, the method of training further comprising performing, prior to step a), by a controller or by the trained machine learning data processing model, image processing to extract the training data set from medical image data. The training data set can be obtained from medical image data using one or more image recognition methods to compute the sequences of luminal dimensions at their corresponding coronary artery locations. The training data set can be fed to the machine learning data processing model in step a).

Optionally, the medical image data is obtained from one or more Computed Tomography, CT, images, and/or Magnetic Resonance Imaging, MRI, images and/or fluoroscopic images.

Optionally, image processing further comprises automatically detecting, by the controller or by the trained machine learning data processing model, the sequence of luminal dimensions from the medical image data. The luminal dimensions can be automatically detected from the medical image data using one or more image recognition data processing algorithms.

Optionally, the ground truth data set includes data representative of at least one of measured FFR, computed FFR and simulated FFR. The measured FFR can be obtained by coronary pressure measurement. The computed and/or simulated FFR can be obtained using a computer system according to the methods for evaluating a cardiac region of a subject from medical data as described. The computed and/or simulated FFR can be obtained under certain conditions by a method of classifying luminal dimensions using a trained machine learning model as described. If the trained machine learning model, used in the method of classifying luminal dimensions, is trained based on ground truth data comprising different FFR values than a computed FFR and/or simulated FFR value obtained by the method of classifying luminal dimensions is associated with, said computed FFR and/or simulated FFR value can e.g. be fed as ground truth data in the training method.

Optionally, the ground truth data is indicative of at least a first and second FFR class by linking the training data to predetermined associated FFR ranges. The first FFR range can include FFR values of 0.8 or greater, and the second FFR range can include FFR values of smaller than 0.8. In the ground truth data, the first FFR class can link the training data associated with the first FFR range to the first FFR class, thereby indicating a relatively low risk of a functionally significant lesion. The second FFR class can link the training data associated with the second FFR range to the second FFR class, thereby indicating a relatively high risk of a functionally significant lesion being present in the vessel.

Optionally, the training data and/or ground truth data includes patient data, such as age, gender, weight and/or length. By including patient data in the training data, the trained machine learning model can also receive patient data as an input. By providing patient data as an input to the trained machine learning model, the corresponding class of FFR values to which the luminal dimensions correspond can be determined more accurately, since more information on the conditions of the luminal dimensions aids in improving the classification thereof.

According to a sixth aspect is provided an electronic image processing system for use in a method of classifying a sequence of luminal dimensions of a coronary artery in order to determine a FFR classification as described. The system comprises a trained machine learning data processing model that is trained as described. The system can include one or more processors, a controller, a communication circuitry, a power supply, a user interface, and/or a memory. The trained machine learning data processing model can be stored on one of the one or more processors and on the memory. The system comprises one or more instructions, which when loaded into the memory enable the processor to perform the steps of the method for evaluating a cardiac region of a subject from medical data and/or the method of classifying a sequence of luminal dimensions of a coronary artery in order to determine a FFR classification as described.

It will be appreciated that any of the aspects, features and options described in view of the method for evaluating a cardiac region of a subject apply equally to the method for assisting in stent selection and the electronic image processing system, and vice versa. It will also be clear that any one or more of the above aspects, features and options can be combined.

### BRIEF DESCRIPTION OF THE DRAWINGS

Embodiments of the present invention will now be described in detail with reference to the accompanying drawings in which:

FIG. 1 shows an illustration of a schematic representation of an example of a system for detecting obstructive coronary artery disease;

FIG. 2 shows an exemplary flow chart of a computer-implemented method for evaluating a cardiac region of a subject from medical data;

FIG. 3 shows an exemplary flow chart of a computer-implemented method for evaluating a cardiac region of a subject from medical data;

FIGS. 4A, 4B and 4C show illustrations of a schematic representation of an example of a three-dimensional coronary model;

FIG. 5 shows an example of a flow chart of a method of classifying a sequence of luminal dimensions of a coronary artery;

FIG. 6 shows an example of a flow chart of a method of training a machine learning data processing model for performing a method of classifying a sequence of luminal dimensions;

FIG. 7 shows an illustration of a schematic representation of an example of a display showing data relating to the FFR of a coronary artery;

FIG. 8 shows an example of a flow chart of a computer-implemented method for assisting in selecting a preferred stent for implantation; and

FIG. 9 shows an illustration of a schematic representation of an example of an electronic image processing system.

### DETAILED DESCRIPTION

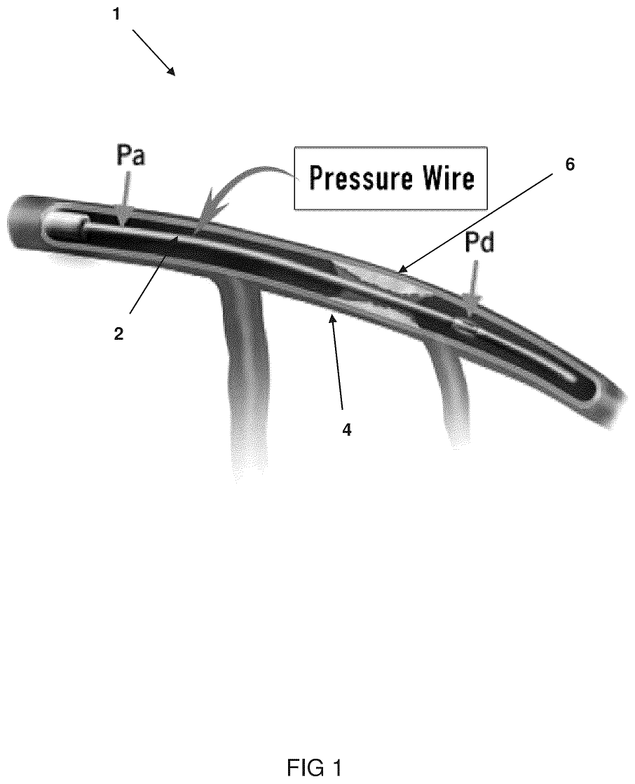

FIG. 1 shows an illustration of a schematic representation of an example of a known system 1 for detecting obstructive coronary artery disease by measuring coronary pressure by inserting a pressure sensor 2, in this example a pressure wire, in a coronary artery 4 of a subject. Prior to the actual measurement, a physician can widen the coronary artery by adding a liquid thereto. The pressure sensor 2 is used to measure the proximal coronary pressure (Pa) and distal coronary pressure (Pd), i.e. the coronary pressure at locations along the coronary artery upstream and downstream of a coronary artery obstruction 6, during maximum hyperemia. The detected proximal and distal coronary pressures are used to determine the Fractional Flow Reserve, FFR, of the coronary artery. The FFR is commonly defined as the ratio of the coronary pressures measured directly distal and proximal of the stenosis 6, i.e., FFR=Pd/Pa. The FFR is a dimensionless number in the numerical range from 0 (complete occlusion) through 1 (no occlusion). An FFR-value below 0.75-0.80 is generally considered to reflect a clinically significant stenosis.

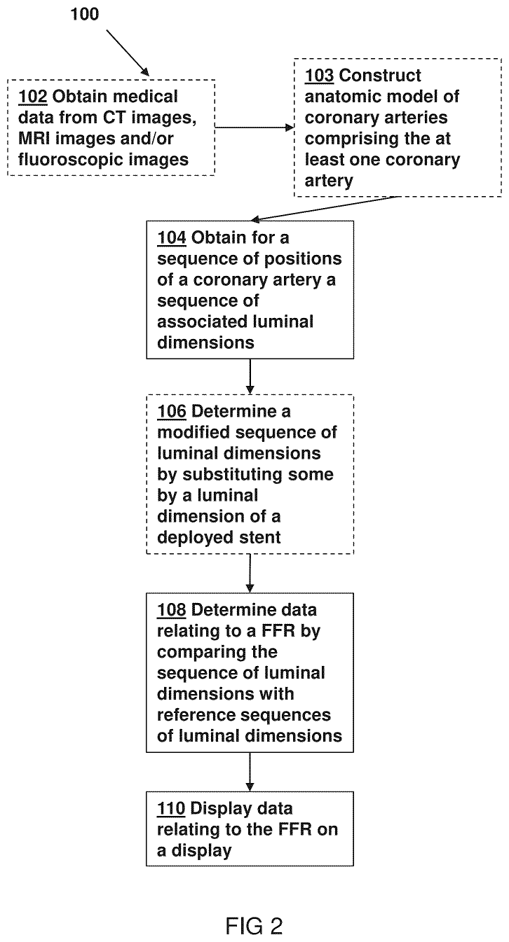

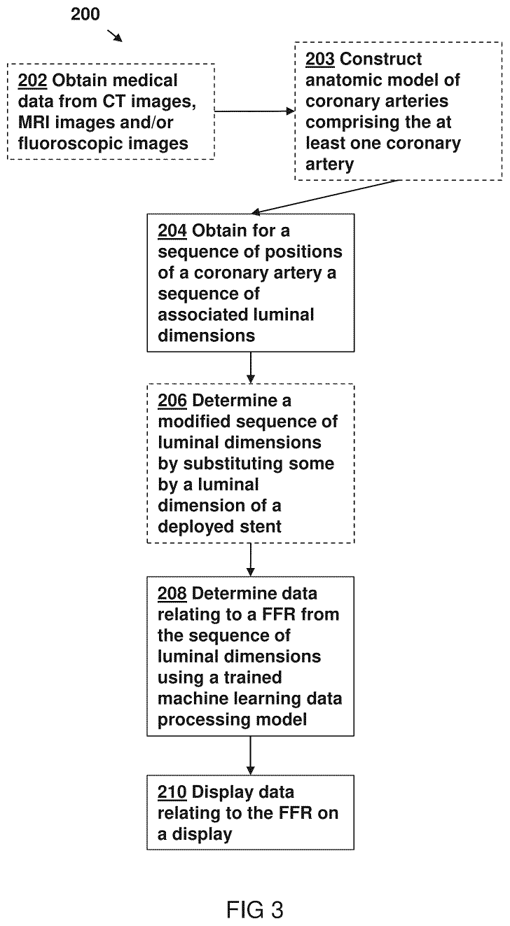

FIGS. 2 and 3 show examples of flow charts of computer-implemented methods 100, 200 for evaluating a cardiac region of a subject from medical data. Optional steps are shown in dashed boxes. In these examples of the methods 100, 200 the steps are performed in the following order. The medical data is in this example obtained from one or more Computed Tomography, CT, images in steps 102, 202. It will be appreciated that the medical data can also be obtained from Magnetic Resonance Imaging, MRI, images and/or fluoroscopic images in steps 102, 202. From the image data of the CT images, an anatomic model of coronary arteries 10, comprising the at least one coronary artery 4, can be constructed in step 103, 203. In step 104 of method 100, for a sequence, such as an array, of positions along the length of the coronary artery, a sequence, such as an array, of associated luminal dimensions is obtained. Here, the sequence of luminal dimensions is determined by the computer system from the medical data. The sequence of luminal dimensions can also be determined from the anatomic model of coronary arteries optionally generated in step 103, 203. Optionally, in steps 106, 206 a modified sequence of luminal dimensions is determined by substituting a determined luminal dimension of the coronary artery by a luminal dimension of a deployed stent. In steps 106 and/or 206, the luminal dimensions for at least some of the positions along the centerline can be substituted by luminal dimensions of a deployed stent at associated positions along the centerline. The modified sequence of luminal dimensions can comprise some original luminal dimensions and some altered luminal dimensions due to computed stent deployment.

The data relating to a FFR of the coronary artery is determined in step 108 by comparing the sequence of luminal dimensions of the coronary artery of the subject with one or more reference sequences of luminal dimensions by the computer system. The reference sequences of luminal dimensions can be obtained from medical data from other coronary arteries of the same subject or from medical data of other subjects. The reference sequences of luminal dimensions can be obtained using a computer system and/or by coronary pressure measurement. If step 106 or 206 is included in method 100 or 200, the data relating to the FFR subsequently determined in step 108 or 208 can predict a value of the data relating to the FFR if said deployed stent would be present in the coronary artery. If step 106 is included, method 100 compares the modified sequence of luminal dimensions to the reference sequences of luminal dimensions in step 108. The modified sequence of luminal dimensions is in method 200 fed to the trained machine learning data processing model in step 208, if step 206 is included, such that a predicted value of the data relating to the FFR is determined.

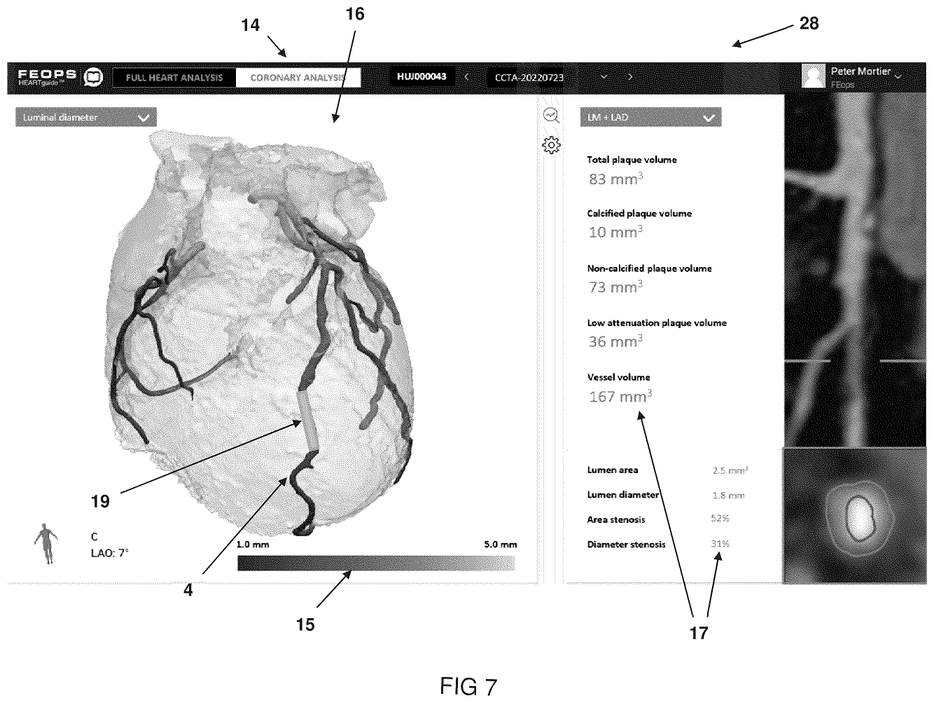

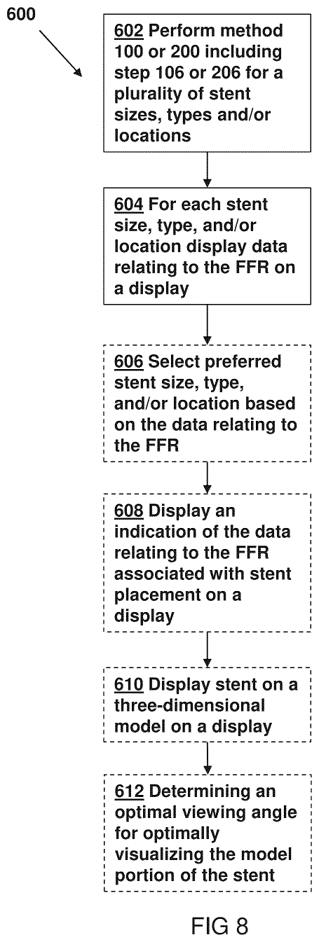

In step 110, the data relating to the FFR is displayed on a display. In step 204 of method 200, for a sequence of positions along a centerline of the coronary artery, a sequence of associated luminal dimensions is obtained by the computer system. The data relating to a FFR of the coronary artery is determined, by the computer system, in step 208 from the sequence of luminal dimensions using a trained machine learning data processing model. The trained machine learning data processing model is trained. A detailed example of a trained machine learning model is described below in view of FIG. 7 and the training thereof is described in view of FIG. 8. In step 210, the data relating to the FFR is displayed on a display.

In this example, determining the data relating to the FFR in the respective method steps 108, 208 comprises determining a single FFR value for the entire sequence of luminal dimensions of the coronary artery. Alternatively, a sequence of progressive or local FFR's is determined for the sequence of luminal dimensions. A progressive or local FFR value can be computed for each coronary artery obstruction, wherein more than one obstruction can occur in a single coronary artery. A progressive or local FFR value can be computed for each position of the sequence of positions along the length of the coronary artery. In this example, the computer system automatically determines the sequence of luminal dimensions from the medical data, i.e. the medical image data. The luminal dimensions comprise one or more of local diameter, average diameter, minimum diameter, maximum diameter, volume, cross sectional area, and curvature radius of the coronary artery. The luminal dimension data can be related to the respective position along the centerline of the coronary artery. The luminal dimension data can also comprise an indication of the presence of plaque. Further, the luminal dimension data can comprise at least one of size, location and severity of the plaque that is present in the coronary artery. The steps 108, 208 of determining data relating to the FFR can comprise using patient data, such as age, gender, weight and/or length, as input.

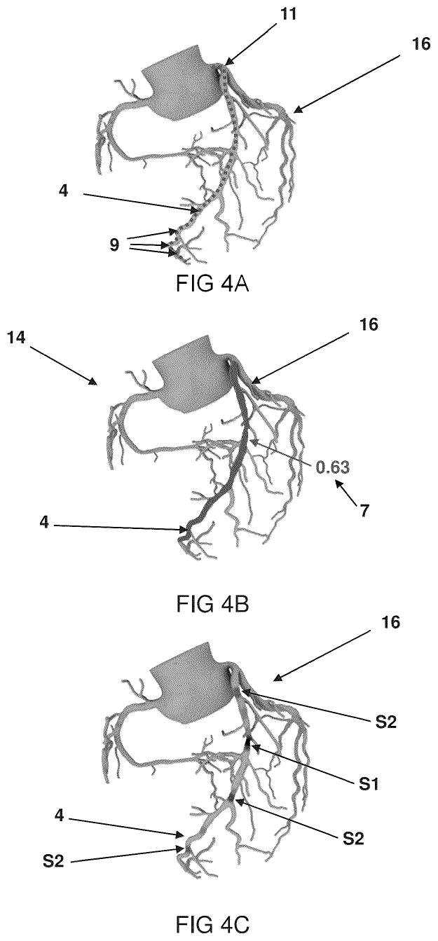

FIGS. 4A, 4B and 4C show illustrations of a schematic representation of an example of a three-dimensional coronary model 16 comprising the coronary artery 4. FIG. 4A shows a three-dimensional coronary model 16 prior to determining the data relating to the FFR. The three-dimensional coronary model 16 can be obtained from a 3D medical image such as a CT image or MRI image. In FIG. 4A, the virtual coronary artery 4 is computationally divided into a plurality of slices 9 along the centerline 11, here shown by a red dotted line, of the coronary artery 4. This can be included in method steps 102,202 and/or 104,204. In this example, each dot of the centerline 11 represents a center of a coronary artery slice 9. Here, the coronary artery 4 is computationally divided into slices 9 at regular intervals. The intervals are evenly distributed, resulting in the slices 9 having an approximately similar thickness. The interval can e.g. be 1 mm, or 0.5 mm. The intervals of the slices 9 together indicate the sequence of positions along the centerline 11 of the coronary artery 4. The luminal dimensions can be computed for each slice 9, thereby resulting in a sequence of luminal dimensions associated with the sequence of positions along the centerline 11. The sequence of luminal dimensions and the sequence of positions can each comprise an array of numerical values that is fed to the computer system in method steps 104, 204.

FIG. 4B shows the three-dimensional coronary model 16 after determining the data relating to the FFR. The data relating to the FFR can be determined using a method 100 and/or 200 as described in view of FIG. 2 or 3. Here, a single FFR value is displayed for the sequence of luminal dimensions of the coronary artery 4. In the example the single value is 0.63. The display 14 of FIG. 4B highlights the coronary artery 4 of which the respective FFR value 7 is displayed. Here, the FFR value is displayed on a three-dimensional coronary model 16 on the display 14.

FIG. 4C shows an example in which a local FFR value is determined for each position of the sequence of positions along the length of the at least one coronary artery. The local FFR value indicates the ratio between the coronary pressures measured directly distal and proximal of the respective location, e.g. directly upstream and downstream of the respective slice. The local FFR can be an indication of a local occlusion. In the example of FIG. 4C, the local FFR values are shown as a false color scale grey scale applied to the anatomical model of the coronary arteries. In FIG. 4C a major occlusion S1 and minor occlusions S2 can be seen. These occlusion sites can provide input for determining where placing a stent might be beneficial.

The methods 100 and/or 200 can further comprise identifying a region of interest in the medical image data, wherein the region of interest comprises coronary arteries highlighted on the three-dimensional coronary model 16. The region of interest can be indicated in a display. The region of interest optionally includes the presence of plaque and/or the obstructed coronary artery.

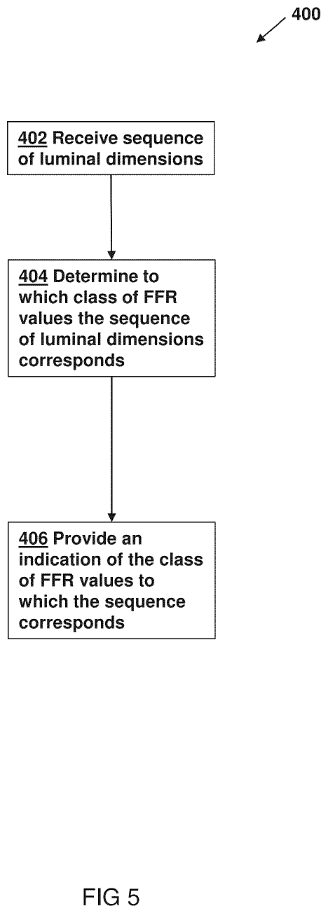

FIG. 5 shows an example of a flow chart of, in an electronic image processing system 30, a method 400 of classifying a sequence of luminal dimensions of a coronary artery 4 in order to determine a FFR classification of the coronary artery of a subject by using a trained machine learning data processing model. Here, the trained machine learning data processing model is trained to classify the sequence of luminal dimensions as representing an FFR value according to a first class of FFR values or a second class of FFR values. The first class can e.g. correspond to FFR values greater than or equal to 0.8, and the second class can e.g. correspond to FFR values smaller than 0.8. The first class can e.g. correspond to FFR values greater than 0.8, and the second class can e.g. correspond to FFR values smaller than or equal to 0.8. Alternatively, the first class can e.g. correspond to non-ischemic patients and the second class can correspond to ischemic patients. It will be appreciated that the trained machine learning model can be trained to classify the FFR values according to more than two classes of FFR values. For instance The first class can e.g. correspond to FFR values greater than (or equal to) 0.85, the second class can e.g. correspond to FFR values between 0.75 and 0.85 and a third class can e.g. correspond to FFR values smaller than (or equal to) 0.75. In these examples of the method 400 the steps are performed in the following order. In a first step 402, the sequence of luminal dimensions is received by the trained machine learning data processing model. It is then determined, using the trained machine learning model, to which of the classes of FFR values the sequence of luminal dimensions corresponds in step 404.

In step 406, an indication of the class of FFR values to which the sequence of luminal dimensions corresponds is provided. In this example, the indication can either comprise an indication of the first class of FFR values or an indication of the second class of FFR values for said coronary artery 4. It will be appreciated that an indication of more possible classes of FFR values can be provided corresponding to the number of classes provided in training of the machine learning model. The sequence of luminal dimensions received by the trained model in step 402 comprises at least one of local diameter, average diameter, minimum diameter, maximum diameter, volume, cross sectional area, and curvature radius of the coronary artery. Further, the sequence of luminal dimensions optionally comprises an indication of the presence of plaque, and at least one of size, location and severity of plaque.

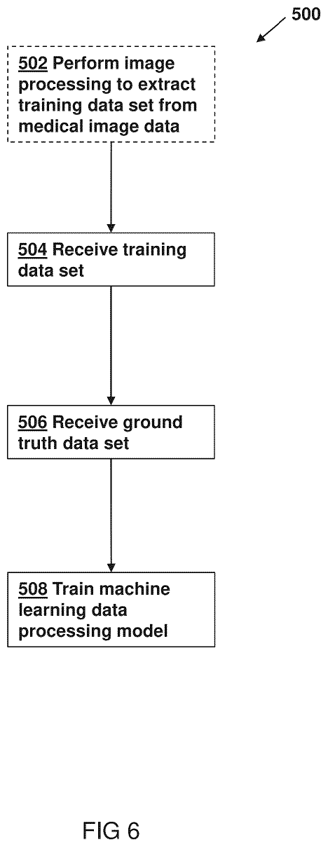

FIG. 6 shows an example of a flow chart of a method 500 of training a machine learning data processing model for performing a method 400 as described, for classifying a sequence of luminal dimensions of a coronary artery in order to determine a FFR classification of the coronary artery of a subject. Optional steps are shown in dashed boxes. In these examples of the method 500 the steps are performed in the following order. Image processing is optionally performed, by a controller 35 or by the trained machine learning data processing model, to extract the training data set from medical image data in step 502. The medical image data is in this example obtained from a plurality of CT images. It will be appreciated that the medical image data can also be obtained from one or more MRI images and/or fluoroscopic images. The training data set can be obtained from medical image data in step 502 using one or more image recognition methods to compute the sequences of luminal dimensions at their corresponding coronary artery locations. Optionally, the image processing step 502 comprises automatically detecting the sequence of luminal dimensions at their coronary artery locations along the centerline from the medical image data, i.e. using one or more image recognition data processing algorithms. The automatic detection of the luminal dimensions can be performed by the trained machine learning data processing model. The training data set is fed to the machine learning data processing model.

In step 504, the training data set is received by the machine learning data processing model. The training data set comprises training data, and the training data includes a plurality of known sequences of luminal dimensions along coronary artery locations. The known sequences of luminal dimensions can be from other coronary arteries of the same subject or from other subjects. Next, a ground truth data set is received by the machine learning data processing model in step 506. The ground truth data set comprises ground truth data indicative of the first and second FFR class associated with the training data in this example. In step 508, the machine learning data processing model is trained. The machine learning model is trained in step 508 based on the training data received in step 504, and the ground truth data received in step 506. The ground truth data set, received in step 506, includes data representative of measured FFR, computed FFR and/or simulated FFR. The measured FFR can be obtained using the system in view of FIG. 1. The computed FFR and/or simulated FFR can be obtained using methods 100,200 and/or under certain conditions 400 in view of FIGS. 2, 3 and/or 5. A computed FFR and/or simulated FFR obtained by method 400 can e.g. be fed as ground truth data in method step 506, if the trained machine learning model used in method 400 is trained based on ground truth data comprising data representative of different FFR values than the computed FFR and/or simulated FFR values are associated with. The training data and/or ground truth data received by the machine learning model in steps 504 and/or 506 can comprise patient data, such as age, gender, weight and/or length, as input.

In this example, the ground truth data is indicative of a first and second FFR class by linking the training data to predetermined associated FFR ranges. In this example the first FFR range includes FFR values of 0.8 or greater, the second FFR range includes FFR values of smaller than 0.8. In the ground truth data, the first FFR class links the training data associated with the first FFR range to the first FFR class, thereby indicating a relatively low risk of a functionally significant lesion. The second FFR class links the training data associated with the second FFR range to the second FFR class, thereby indicating a relatively high risk of a functionally significant lesion being present in the vessel.

Here, the machine learning data processing model is trained in step 508 for enabling said machine learning model, after completion of the training period, to perform the step of classifying the sequence of luminal dimensions as representing an FFR value according to the first class of FFR values or the second class of FFR values. If the sequence of luminal dimensions is classified according to the first FFR class, this implies a relatively low risk of a significant obstruction being present in the vessel. Conversely, from a classification of a sequence of luminal dimensions according to the second FFR class follows a significant risk of a significant obstruction being present in the vessel. An indication of the FFR class of a coronary artery can aid the physician in evaluating a possible obstruction of a coronary artery, due to the FFR range and diagnostic accuracy information provided therewith.

FIG. 7 shows an illustration of a schematic representation of an example of a user interface 28 displaying a three-dimensional coronary model 16 and showing data relating to the FFR of a coronary artery 4 of a subject. The data relating to the FFR can be determined using any of computer-implemented methods 100,200,400 and/or 500 as described in view of FIGS. 2, 3,, 5 and/or 6. The difference in luminal dimensions is in this example shown in the colored part of the coronary artery 4, here in a false color scale 15. Here, the three-dimensional coronary model 16 and the user interface 28 are displayed on a display 14, e.g. for use during examination of a subject by a physician.

The parameter values 17 resulting from the computations during performing of any of the methods 100,200,400 and/or 500 are displayed on the display 14 in this example. It will be appreciated that the luminal dimensions and/or data relating to the FFR can be displayed in different forms as well. Here, the luminal dimensions are also shown numerically, here as an absolute value, at 17. In this example, the parameter values are total plaque volume, calcified plaque volume, non-calcified plaque volume, low attenuation plaque volume, vessel volume, lumen area, lumen diameter, stenosis area and stenosis diameter. Optional parameters can be aortic valve calcification, ascending aorta diameter, porcelain aorta, aortic valve morphology, mitral valve calcification, cardiomyopathy and myocardial calcification. Here, the potential placement of a stent 19 according to method 600 in view of FIG. 8 is shown on a preferred coronary artery 4 location on the three-dimensional coronary model 16.

FIG. 8 shows an example of a flow chart of a computer implemented method 600 for assisting in selecting a preferred stent 19 for implantation. It will be appreciated that implantation in this case refers to virtual stent implantation, as it concerns a computer implemented method. The virtual implantation can be used to simulate implantation of the stent in the model of the patient's anatomy. Hence, efficacy of stent placement can be simulated without surgical intervention to the patient. This allows multiple stent types, sizes and/or locations to be tested without hardship to the patient. Optional steps are shown in dashed boxes. In these examples of the method 600 the steps are performed in the following order. In step 602, the method 100 or 200 including step 106 or 206 is performed for a plurality of stent sizes, types, and/or locations in the coronary artery 4. For each of the plurality of stent sizes, types, and/or locations, the data relating to the FFR is displayed in step 604 on a display 14. Optionally, in step 606 a preferred stent size, type, and/or location is selected by the computer. The preferred stent size, type, and/or location can be selected based on the data relating to the FFR for each of the plurality of stent sizes, types, and/or locations determined in step 602. For a relatively high FFR value, the implications from computing stent deployment result in relatively low modifications in luminal dimensions.

In step 604, the method 100 or 200 including step 106 or 206 is in this example performed for a single stent size and type, for placement of the stent 19 in the coronary artery 4 at a plurality of different locations along the entire sequence of positions 9 along the centerline 11. Here, stent placement refers to the end location of the stent, i.e. the position of the stent after deployment in the coronary artery. The placement of the stent here does not refer to the process of moving the stent into the subject to the desired position. Stent placement is therefore concerning a final stent destination. An indication representative of a value of the data relating to the FFR associated with placement of the stent 19 at said locations along the sequence of positions is optionally displayed on a display in step 608. It will be appreciated that for a single stent type and location, the method 100 or 200 can be performed in step 602 for multiple different sizes of stents. Also, for a single stent size and location, the method 100 or 200 can be performed for multiple different types of stents in step 602. Optionally, a three-dimensional model of the stent 19 is displayed on a three-dimensional coronary model 16 on the display 14.

Steps 608 and 610 can comprise showing multiple scenario's of stent positions in multiple windows on the display 14, each window comprising a three-dimensional model 16. Alternatively, multiple stent positions can be incorporated in one window on the same three-dimensional model. In step 610 also image features and/or parameter values can be displayed on the display 14. Optionally, the view and/or displayed parameter values of the coronary model are manipulated in step 610. This can be done by a user through for example a user interface 28. Alternatively, the view and/or displayed parameter values of the coronary model can be changed automatically according to the stent number, size and/or location.

In step 612, an optimal viewing angle of the three-dimensional coronary model for optimally visualizing the stent portion of the three-dimensional coronary model associated with the placement of the stent can be determined. In this example, the optimal viewing angle can correspond to an optimal c-arm position for CT imaging of the cardiac region of the subject, e.g. during actual insertion of an actual stent in the patient's body. Step 612 optionally comprises recording a position of a C-arm of an X-ray imaging unit that corresponds to the optimal viewing angle. Further, the position of the C-arm corresponding to the optimal viewing angle can be established automatically.

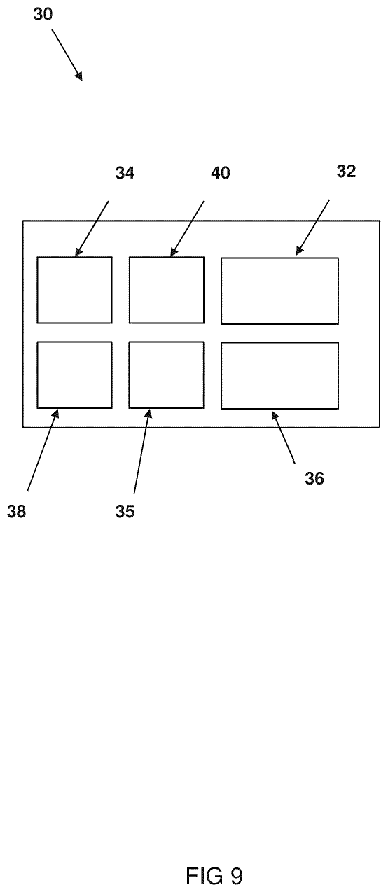

FIG. 9 shows an illustration of a schematic representation of an example of an electronic image processing system 30 for use in a method 400 as described in view of FIG. 5. The system 30 comprises a trained machine learning data processing model that is trained using method 500 as described in view of FIG. 6. The system 30 further comprises a processor 32 and a memory 34 storing the trained machine learning data processing model and one or more instructions, which when loaded into the memory 34 enable the processor 32 to perform the steps of methods 100, 200, 400 and/or 500.

System 30 may include one or more processors 32, controller 35, communication circuitry 36, power supply 38, user interface 40, and/or memory 34. One or more electrical components and/or circuits may perform some of or all the roles of the various components described herein. Although described separately, it is to be appreciated that electrical components need not be separate structural elements. For example, system 30 and communication circuitry 36 may be embodied in a single chip. In addition, while system 30 is described as having memory 34, a memory chip(s) may be separately provided.

System 30 may contain memory and/or be coupled, via one or more buses, to read information from, or write information to, memory. Memory 34 may include processor cache, including a multi-level hierarchical cache in which different levels have different capacities and access speeds. The memory may also include random access memory (RAM), other volatile storage devices, or non-volatile storage devices. Memory 34 may be RAM, ROM, Flash, other volatile storage devices or non-volatile storage devices, or other known memory, or some combination thereof, and preferably includes storage in which data may be selectively saved. For example, the storage devices can include, for example, hard drives, optical discs, flash memory, and Zip drives. Programmable instructions may be stored on memory 34 to execute algorithms for identifying anatomical landmarks in medical images, e.g., MSCT, generating virtual 3D models of anatomical structures, and deriving measurements of the identified anatomical landmarks and structures.

System 30 may incorporate processor 32, which may consist of one or more processors and may be a general purpose processor, a digital signal processor (DSP), an application specific integrated circuit (ASIC), a field programmable gate array (FPGA) or other programmable logic device, discrete gate or transistor logic, discrete hardware components, or any suitable combination thereof designed to perform the functions described herein. System 30 also may be implemented as a combination of computing devices, e.g., a combination of a DSP and a microprocessor, a plurality of microprocessors, one or more microprocessors in conjunction with a DSP core, or any other such configuration.

System 30, in conjunction with firmware/software stored in the memory may execute an operating system, such as, for example, Windows, Mac OS, Unix or Solaris 5.10. System 30 also executes software applications stored in the memory. For example, the software may include, Unix Korn shell scripts, and/or may be programs in any suitable programming language known to those skilled in the art, including, for example, C++, PHP, or Java.

Communication circuitry 36 may include circuitry that allows system 30 to communicate with an image capture device and/or other computing devices for receiving image files, e.g., MSCT. Additionally or alternatively, image files may be directly uploaded to system 30. Communication circuitry 36 may be configured for wired and/or wireless communication over a network such as the Internet, a telephone network, a Bluetooth network, and/or a WiFi network using techniques known in the art. Communication circuitry 36 may be a communication chip known in the art such as a Bluetooth chip and/or a WiFi chip. Communication circuitry 36 permits system 30 to transfer information, such as 3D model reconstructions and measurements, locally and/or to a remote location such as a server.

Power supply 38 may supply alternating current or direct current. In direct current embodiments, power supply may include a suitable battery such as a replaceable battery or rechargeable battery and apparatus may include circuitry for charging the rechargeable battery, and a detachable power cord. Power supply 38 may be charged by a charger via an inductive coil within the charger and an inductive coil within the power supply. Alternatively, power supply 6 may be a port to allow system 30 to be plugged into a conventional wall socket, e.g., via a cord with an AC to DC power converter and/or a USB port, for powering components within system 30.

User interface 40 may be used to receive inputs from, and/or provide outputs to, a user. For example, user interface 40 may include a touchscreen, display, switches, dials, lights, etc. Accordingly, user interface 40 may display information such as 3D model reconstructions, measurements, and/or simulations. Moreover, user interface 40 may receive user input. In some embodiments, user interface 40 is provided on a remote, external computing device communicatively connected to system 30 via communication circuitry 36.

Memory 34, which is one example of a non-transitory computer-readable medium, may be used to store operating system, and modules provided in the form of computer-executable instructions that may be executed by processor 32 for performing various operations in accordance with the disclosure.

Herein, the invention is described with reference to specific examples of embodiments of the invention. It will, however, be evident that various modifications and changes may be made therein, without departing from the essence of the invention. For the purpose of clarity and a concise description features are described herein as part of the same or separate embodiments, however, alternative embodiments having combinations of all or some of the features described in these separate embodiments are also envisaged.

However, other modifications, variations, and alternatives are also possible. The specifications, drawings and examples are, accordingly, to be regarded in an illustrative sense rather than in a restrictive sense.

For the purpose of clarity and a concise description features are described herein as part of the same or separate embodiments, however, it will be appreciated that the scope of the invention may include embodiments having combinations of all or some of the features described.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage.

1. A computer-implemented method for evaluating a cardiac region of a subject from medical data, the method comprising the steps of:
a) obtaining, by the computer system, for a sequence of positions along a centerline of the coronary artery a sequence of associated luminal dimensions;
b1) determining, by the computer system, data relating to a Fractional Flow Reserve, FFR, of the coronary artery by comparing the sequence of luminal dimensions of the coronary artery of the subject with one or more reference sequences of luminal dimensions; and
c) displaying the data relating to the FFR on a display.

2. A computer-implemented method for evaluating a cardiac region of a subject from medical data, the method comprising the steps of:
a) obtaining, by the computer system, for a sequence of positions along a centerline of the coronary artery a sequence of associated luminal dimensions;
b2) determining, by the computer system, data relating to a Fractional Flow Reserve, FFR, of the coronary artery from the sequence of luminal dimensions using a trained machine learning data processing model, wherein the trained machine learning data processing model is trained to determine data relating to an FFR based on a sequence of luminal dimensions; and
c) displaying the data relating to the FFR on a display.

3. The method according to claim 1 or 2, wherein a single FFR is determined for the sequence of luminal dimensions.

4. The method according to claim 1 or 2, wherein a sequence of local FFR's is determined for the sequence of luminal dimensions.

5. The method according to any of the preceding claims, wherein the luminal dimensions comprise one or more of diameter, average diameter, minimum diameter, maximum diameter, volume, cross sectional area, and curvature radius of the coronary artery.

6. The method according to claim 5, wherein the luminal dimensions further comprise an indication of the presence of plaque and at least one of size, location and severity of the plaque.

7. The method according to any of the preceding claims, wherein the step of determining data relating to the FFR includes patient data, such as age, gender, weight and/or length, as input.

8. The method according to any of the preceding claims, further comprising, e.g. automatically, determining, by the computer system, the sequence of luminal dimensions from medical image data.

9. The method according to claim 8, wherein the medical image data is obtained from one or more Computed Tomography, CT, images, Magnetic Resonance Imaging, MRI, images and/or fluoroscopic images.

10. The method according to any of the preceding claims, further comprising determining, prior to step b1 or b2, a modified sequence of luminal dimensions, by substituting, for at least some of the positions along the centerline, a determined luminal dimension of the coronary artery by a luminal dimension of a deployed stent.

11. A computer implemented method for assisting in selecting a preferred stent for implantation, comprising:
d) for a plurality of stent sizes, types, and/or locations in a coronary artery, performing the method of claim 10; and
e) for each of the plurality of stent sizes, types, and/or locations displaying the data relating to the FFR on a display.

12. The method of claim 11, further comprising the computer selecting a preferred stent size, type, and/or location on the basis of the data relating to the FFR for each of the plurality of stent sizes, types, and/or locations determined in step d).

13. The method of claim 11 or 12, comprising:
for a single stent size and type, performing the method of claim 10 for placement of the stent in the coronary artery at a plurality of different locations along the entire sequence of positions along the centerline; and
displaying an indication representative of a value of the data relating to the FFR associated with placement of the stent at said locations along the sequence of positions on a display.

14. The method of any of claims 10-13, further comprising displaying the stent on a three-dimensional coronary model on the display.

15. The method according to claim 14, wherein the displaying further comprises displaying image features and/or parameter values on the display.

16. The method according to claim 15, further comprising manipulating the view and/or displayed parameter values of the coronary model.

17. The method according to claim 15 or 16, further comprising automatically changing the view and/or displayed parameter values of the coronary model according to the stent number, size and/or location.

18. The method according to any of claims 14-17, comprising determining an optimal viewing angle of the three-dimensional coronary model for optimally visualizing the portion of the three-dimensional coronary model associated with the placement of the stent.

19. The method according to claim 18, comprising recording a position of a C-arm of a X-ray imaging unit that corresponds to the optimal viewing angle.

20. The method according to claim 19, wherein the position of the C-arm corresponding to the optimal viewing angle is established automatically.

21. In an electronic image processing system, a method of classifying a sequence of luminal dimensions of a coronary artery in order to determine a Fractional Flow Reserve, FFR, classification of the coronary artery of a subject by using a trained machine learning data processing model, wherein the trained machine learning data processing model is trained to classify the sequence of luminal dimensions as representing an FFR value according to at least a first class of FFR values or a second class of FFR values; the method comprising in the following order the steps of:
receiving, by the trained machine learning data processing model, the sequence of luminal dimensions;
determining, using the trained machine learning data processing model, to which of the at least the first and second class of FFR values the sequence of luminal dimensions corresponds;
providing an indication of the class of FFR values to which the sequence of luminal dimensions corresponds.

22. The method according to claim 21, wherein the sequence of luminal dimensions comprises at least one of diameter, average diameter, minimum diameter, maximum diameter, volume, cross sectional area, and curvature radius of the coronary artery, and optionally an indication of the presence of plaque and at least one of size, location and severity of plaque.

23. A method of training a machine learning data processing model for performing a method according to claim 21, for classifying a sequence of luminal dimensions of a coronary artery in order to determine a FFR classification of the coronary artery of a subject, the method comprising:
a) receiving, by the machine learning data processing model, a training data set comprising training data, the training data including a plurality of known sequences of luminal dimensions along coronary artery locations;
b) receiving, by the machine learning data processing model, a ground truth data set comprising ground truth data indicative of at least a first or second FFR class associated with the training data;
c) training the machine learning data processing model based on the training data received in step a), and the ground truth data received in step b), for enabling the machine learning data processing model, after completion of the training period, to perform the step of classifying the sequence of luminal dimensions as representing an FFR value according to one of at least a first class of FFR values or a second class of FFR values.

24. The method according to claim 23, further comprising performing, prior to step a), by a controller or by the trained machine learning data processing model, image processing to extract the training data set from medical image data.

25. The method according to claim 24, wherein the medical image data is obtained from one or more Computed Tomography, CT, images, Magnetic Resonance Imaging, MRI, images and/or fluoroscopic images.

26. The method according to claim 24 or 25, wherein image processing further comprises automatically detecting, by the controller or by the trained machine learning data processing model, the sequence of luminal dimensions from the medical image data.

27. The method according to any of claims 23-26, wherein the ground truth data set includes data representative of at least one of measured FFR, computed FFR and simulated FFR.

28. The method according to any of claims 23-27, wherein the ground truth data is indicative of at least a first and second FFR class by linking the training data to predetermined associated FFR ranges.

29. The method according to any of claims 23-28, wherein the training data and/or ground truth data includes patient data, such as age, gender, weight and/or length.

30. An electronic image processing system for use in a method according to any of claims 21-22, the system comprising a trained machine learning data processing model according to any of claims 24-26.
